SEQUENCE LISTING (6 sequences)

SEQ ID NO: 1 - protein, 360 aa, synthetic construct
MIILIFLIFSNIVLSIDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVRYTNESILEYNWNNSNINNFTATCIINNTISTSNETTLINCTYLTLSSNYFYTFFKLYYIPLSIIIGITISILLISIITFLSLRKRKKHVEEIESPPPESNEEEQCQHDDTTSIHEPSPREPLLPKPYSRYQYNTPIYYMRPSTQPLNPFPLPKPCPPPKPCPPPKPCPPPKPCPSAESYSPPKPLPSIPLLPNIPPLSTQNISLIHVDRII

SEQ ID NO: 2 - protein, 220 aa, synthetic construct
IDYWVSFNKTIILDSNITNDNNDINGVSWNFFNNSFNTLATCGKAGNFCECSNYSTSIYNITNNCSLTIFPHNDVFDTTYQVVWNQIINYTIKLLTPATPPNITYNCTNFLITCKKNNGTNTNIYLNINDTFVKYTNESILEYNWNNSNINNFTATCIINNTISTSNETTLINCTYLTLSSNYFYTFFKTFFKLYYIPLSIIIGITISILLISIITFLSL

SEQ ID NO: 3 - protein, 158 aa, synthetic construct
MFSNKKYIGLINKKEGLKKKIDDYSILIIGILIGTNILSLIINIIGEINKPICYQNDDKIFYCPKDWVGYNNVCYYFGNEEKNYNNASNYCKQLNSTLTNNNTILVNLTKTLNLTKTYNHESNYWVNYSLIKNESVLLRDSGYYKKQKHVSLLYICSK

SEQ ID NO: 4 - protein, 113 aa, synthetic construct
GEINKPICYQNDDKIFYCPKDWVGYNNVCYYFGNEEKNYNNASNYCKQLNSTLTNNNTILVNLTKTLNLTKTYNHESNYWVNYSLIKNESVLLRDSGYYKKQKHVSLLYICSK

SEQ ID NO: 5 - protein, 183 aa, synthetic construct
MDSEFFQPVYPRHYGECLSPVTTPSFFSTHMYTILIAIVVLVIIIIVLIYLFSSRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASTMSAIENLRQRNTYTHKDLENSL

SEQ ID NO: 6 - protein, 130 aa, synthetic construct
SRKKKAAAIEEEDIQFINPYQDQQWVEVTPQPGTSKPAGATTASVGKPVTGRPATNRPATNKPVTDNPVTDRLVMATGGPAAAPAAASAPAHPAEPYTTVTTQNTASTMSAIENLRQRNTYTHKDLENSL

(19) United States
(12) Patent Application Publication
(10) Pub. No.: US 2026/0335423 A1
(43) Pub. Date: Oct. 29, 2026

(54) DOMINANT NEGATIVE ANTIGEN APPROACH FOR PROPHYLACTIC AND POST-INFECTION TREATMENT OF SWINE AGAINST AFRICAN SWINE FEVER VIRUS

(71) Applicants: Thomas MALCOLM, Andover, NJ (US); Dalu CHEN, Erie, CO (US)

(72) Inventors: Thomas MALCOLM, Andover, NJ (US); Dalu CHEN, Erie, CO (US)

(21) Appl. No.: 19/038,886

(22) PCT Filed: Aug. 4, 2023
(86) PCT No.: PCT/US2023/071659

§ 371 Date: Jan. 28, 2025

Related U.S. Application Data
(60) Provisional application No. 63/395,275, filed on Aug. 4, 2022.

(51) Int. Cl. A61K 39/12; C07K 14/005

(52) U.S. Cl. CPC A61K 39/12; C07K 14/005; C12N 2710/12022; C12N 2710/12034

(57) ABSTRACT

The disclosed invention pertains and encompasses a composition comprising modified ASFV outer-membrane protein antigen mutants (termed dominant negatives) that exhibit non-binding affinity to RBCs while inducing an antibody-mediated response capable of neutralizing unmodified proteins found on infectious outer-membrane-laden ASFV virions. Additionally, a method for the treatment and/or prevention of ASFV is provided, involving the administration of a dominant negative antigenic composition to animals, thereby averting RBC aggregation caused by the antigen and concurrently treating and/or preventing ASFV. Furthermore, the invention encompasses an ASFV vaccine composition containing the dominant negative antigen as a constituent. Moreover, the invention covers a formulation incorporating these dominant negative antigens in conjunction with antigens derived from capsid-based proteins, which collectively target both lysogenic and lytic viral replication cycles, thereby achieving optimal immune stimulatory protection.

### BACKGROUND OF THE INVENTION

### 1. Technical Field

The present invention relates to compositions and methods for treating and preventing African Swine Fever Virus (ASFV).

### 2. Background Art

African Swine Fever Virus is a large double-stranded DNA virus of the Asfarviridae family that primarily infects domestic pigs, wild boars, warthogs, and bush pigs. It also resides in soft ticks, thereby acting as an infectious vector. ASFV primarily infects the monocytes and macrophages, although, at acute infection many other cell types can be infected. ASFV causes high fever, hemorrhagic lesions, cyanosis, anorexia, and fatalities in these animals. There is no vaccine or treatment for this virus, and the only way to currently prevent its spread is culling animals.

Development of a vaccine and/or treatment for ASFV is a highly active endeavor across multiple laboratories in China, the United States and Europe. There have been many approaches to develop such vaccines/treatments, however, all of them have fallen short of curative/prophylactic expectations.

Some of these approaches have included the engineering of various types of attenuated viruses, immune stimulation using viral antigens in various and multiple combinations, gene editing, and antibody neutralization therapies, to name a few.

Traditional vaccine and therapeutic development protocols mostly take into consideration viral proteins that are exposed on the surface of the virion as potential targets for immune neutralization or as antigens to stimulate the immune system. Other approaches can target viral machinery that is biochemically active within the infected host cell, with small molecule, RNA interfering, or CRISPR gRNA inhibition methods. Further, more exotic approaches such as gene editing (CRISPR for example) can be utilized to alter the viral genome once it has taken root in the infected host cell (Hübner et al., Borca, et al., Woźniakowski et al.).

In the context of ASFV, many of these approaches are unrealistic due to the exorbitantly high cost to individually treat a livestock animal with a relatively low capital value, per head.

In addition to logistic shortcomings, many of the failures of vaccine or therapeutic design can be attributed to a lack of deep understanding of the virus structure and viral replication cycle. Recently, the structure of ASFV has been defined (Liu, et al.). It has since become clear that the virus exists in two forms: 1) A virion capsid surrounded by an outer lipid bilayer and, 2) a naked virion encompassing a capsid only (FIG. 1A). The outer lipid bilayer membrane has been shown to contain human proteins, thereby strongly suggesting that the capsid-based virion buds from the infected cell. This property is mostly associated with the lysogenic replication cycle that is observed in most other viruses. Further, it has been reported that ASFV capsid-based virions (without the outer lipid bilayer membrane) increase in the circulating serum at later stages of infection, strongly correlating to the dynamics of the lytic cycle of most other viruses (see description of lysogenic and lytic ASFV in PCT/US20/50939 to Chen, et al.). Taking these structural components into consideration and thereby defining them in the context of an early lysogenic versus late lytic replication cycle, has allowed Applicants to pursue a novel vaccine and therapeutic development program (PCT/US20/50939, U.S. patent application Ser. No. 17/535,545) (FIGS. 1B and 1C).

Such a strategy takes into consideration the necessity for an immediate neutralization of early infectious membrane bound virions that are derived from a lysogenic cycle, followed by the rapid neutralization of capsid-based virions that exist in low quantities prior to a lytic cycle switch.

Through this strategy, Applicants have tested several known outer-membrane-bound proteins as potential targets for both antibody therapeutic production and antigen-based vaccines. These targets include the outer-membrane proteins EP402R (CD2v), and EP153R, the outer-membrane and inner envelope protein 016R (p12), the inner envelope and capsid spanning protein E183L (p54) as well as several main capsid proteins including B646L (p72), E120R (p14.5), B438L (p49) and CP204L (p30), although research is not limited to these proteins (FIGS. 2 and 3). It has been shown that immune responses against outer-membrane proteins (either as a protein subunit antigen, full protein antigen, or expressed on the surface of an attenuated virus) produce very weak immune responses in swine, likely due to their ability to bind to RBCs, burrow deep within them and cause them to agglutinate. Therefore, this agglutination ability must be eliminated in order to expose the surfaces of outer-membrane antigens including EP402R, EP153R and 016R (but not limited to these protein antigens) to the swine immune system to mount a strong and neutralizing immune reaction (FIG. 4).

The capsid-based proteins have thus far not been shown to cause agglutination in RBCs, although the Applicants have observed a mild swine RBC agglutination effect that is species-specific with the DBD containing and capsid interacting subunit of the inner envelope and capsid spanning protein EL83L (p54) (FIG. 5B). As such, E183L mutations may be necessary to block this ability and expose this antigen effectively to the swine immune system to mount a strong neutralizing effect.

Since the capsid-based proteins have not been shown to have these agglutination properties, dominant negative mutation to achieve strong neutralizing effects may not be necessary. However, combinations of capsid antigens with the dominant negative antigens will likely improve immunogenicity against each stage of the ASFV replication cycle, thereby enforcing the Applicants' thesis that targeting both the lysogenic cycle proteins (mainly outer-membrane proteins) and lytic cycle (mainly capsid-based proteins) will be necessary to effectively protect the swine from ASFV infection. Thus, capsid-based antigen protein(s) in combination with dominant negative outer-membrane proteins such as EP402R, EP153R, and 01 6R, as well as inner envelope and capsid spanning proteins like E183L will be used in combination to stimulate and immune response to effectively neutralize AFSV infection in swine.

Not to be bound by theory, the dominant negative approach to vaccine development for stronger immune reactions has been achieved previously by similar approaches such as the tetanus and diphtheria toxoids. These toxoids are inactivated forms of the toxic proteins for tetanus and diphtheria that produce an effective immune response.

By creating dominant negative protein subunit versions of the wild type ASFV outermembrane protein antigen typically used in vaccines that cause agglutination, the agglutination will not occur, thus exposing the antigens to the immune system (FIG. 4)

Applicants' latest research has shown that the subunits of the outer-membrane target EP402R are most likely responsible for binding the virus to swine red blood cells (RBCs) causing them to agglutinate in species-specific manner (compared to human, murine, and bovine RBCs). (FIG. 5A). Further, the outer-membrane protein EP153R has previously been shown to co-facilitate the agglutination of RBCs alongside EP402R. The Applicants observe EP153R subunit-driven agglutination of RBCs. However, in the presence of the extraluminal domain of EP402R enhanced agglutination occurs. A similar observation using attenuated virus was reported by Petrovan et al 2021. The aggregated RBCs then trigger the natural macrophage to initiate the RBC aggregate destruction through a yet to be determined mechanism. As the RBCs attract macrophage, the virions that are buried within the RBC aggregate are also internalized into the macrophage, the choice cell for infection. There is likely a mechanism that links one of the outer-membrane proteins to RBC aggregation and the internalization into macrophage. Since ASFV induces hemagglutination and the subsequent hemadsorption to the surface of macrophage prior to infection, the role of these proteins in an orchestrated internalization process is highly probable (Malmquist, et al., Yang, et al.). To date, there has never been an ASFV receptor identified on the surface of macrophage that would facilitate a receptor mediated internalization of the virus. However, the uptake of capsid-based virion has been shown through the far less efficient macropinocytosis entry pathway (Sánchez, et al.). For capsid-based virions to efficiently infect macrophage via macropinocytosis on a systemic and rapid level, there must be copious amount of virus circulating in the swine. This likely occurs after the lysogenic replication phase reaches a critical point, and switches to a lytic phase where large amounts of virus are released from the cell. Thus, by neutralizing the proteins responsible for RBC aggregation and preventing the lysogenic cycle, any remaining capsid-based virus is limited to macrophage entry via macropinocytosis and subsequently locked into an early lysogenic phase that are neutralized upon viral budding, as its predecessors (FIG. 6). Finally, remaining capsid-based virions can be neutralized by antibodies derived from capsid-based targets, such as B646L (p72), E120R (p14.5), B438L (p49) and CP204L (p30), thereby eliminating even the smallest threat (FIGS. 6 and 7—replication cycles and strategy overview).

Applicants' previous data has shown that antibodies raised against the outer-membrane protein EP402R prevents and destabilizes RBC aggregates (FIG. 8A) and in a species-specific manner (FIG. 8B). This antibody mediated disruption of the RBC aggregate likely prevents the internalization of these proteins into the macrophage.

As mentioned above, there have been many attempts to use ASFV protein antigens to stimulate an internal antibody response in swine. These attempts have failed to produce any long lasting or meaningful protective measures against the virus in swine challenge models. For example, nearly every capsid protein has been explored as a potential vaccine candidate. With new knowledge of the replication cycles and structure of ASFV (FIGS. 1A-1C and 7), it is not surprising that these approaches have failed. Once the virus switches to a lytic cycle (a critical and populous point after the lysogenic cycle), the immune system or any other protective measure cannot keep up with the overwhelming amount of capsid-based virus. Furthermore, targeting the outer-membrane proteins for antigen-based vaccines has also failed, likely due to their ability to aggregate RBCs and ‘hide’ from B and T-cell responses. For example, EP402R (termed HA in early publications) and EP153R have been shown to facilitate the binding of virus to RBCs, allowing the virus to tunnel/burrow into the RBC membranes. This allows the virus to hide by preventing the exposure of critical surface epitopes from a strong and sustained antibody mediated immune response (Qunitero, et al., Ruiz-Gonzalvo et al.). Noting as well, that several of the outer-membrane proteins also have immune suppressive properties against T-cell responses (Teklue, et al., Petrovan, et al.).

In addition to hemagglutination, EP153R has been shown to sequester MHC class I complexes from being expressed on the surface of T-cells. The amino acid at position Arg133 is critical for the sequestering of MHC Class I complexes after macrophage infection.

These observations have recently been accentuated by observations showing that most of the outer-membrane proteins in attenuated viruses or antigens have relatively weak immune stimulation properties, where exposed capsid proteins have relatively high immune stimulatory characteristics. Therefore, our observation that the outer-membrane proteins EP402R (CD2v), and EP183L (p54) cause RBC aggregation, and previous observations in the literature where EP153R causes lymphocyte neutralizations, occurs specifically in swine and no other species, likely contributes to this synopsis. Furthermore, it is likely that antigen-based vaccine approaches to stimulate an immune response to outer-membrane protein targets fail due to their RBC agglutination properties allowing them to hide from the immune response. Indeed, it is observed that the minimal extraluminal domains of these outer-membrane proteins retain these RBC binding properties (FIGS. 5 A and B).

Therefore, there remains a critical need to develop an antigen-based vaccine using ASFV outer-membrane protein antigens that do not cause RBC aggregation and therefore do not ‘hide’ from eliciting a strong antibody neutralizing immune response.

### SUMMARY OF THE INVENTION

The present invention provides for a composition including engineered ASFV outer-membrane protein antigen mutants (dominant negatives) that do not bind to RBCs yet elicit an antibody response that neutralizes wild type proteins present on infectious outer-membrane containing ASFV virions.

The present invention provides for a method of treating and/or preventing ASFV by administering a composition of a dominant negative antigen to an animal, preventing RBC aggregation from the antigen, and treating and/or preventing ASFV.

The present invention provides for a composition of a vaccine for ASFV, including a dominant negative antigen in a vaccine.

The present invention provides for a composition to use these dominant negative antigens in combination with antigens derived from capsid-based proteins, to address both the lysogenic and lytic viral replication cycles to achieve maximum immune stimulatory protection.

### DESCRIPTION OF THE DRAWINGS

Other advantages of the present invention are readily appreciated as the same becomes better understood by reference to the following detailed description when considered in connection with the accompanying drawings wherein:

FIG. 1A shows the structure of ASFV derived from the lysogenic replication cycle (left) and the subsequent lytic replication cycle (right), FIG. 1B shows why current neutralizing strategies do not work for both replication cycles, and FIG. 1C shows the major defined proteins that exist in the membrane of ASFV virions derived from the lysogenic replication cycle (top) vs. the major defined capsid proteins from virions derived from the lytic replications cycle (bottom);

FIG. 2 shows the legend of polyclonal antibodies that have been raised against the parallel ASFV proteins for pre-clinical studies;

FIG. 3 shows the overall strategy for neutralizing each type of ASFV (with or without membrane) by neutralizing antibodies produced after antigen injection;

FIG. 4 is a representation showing why the dominant negative effect will work far more effectively than using wild type protein or subunit antigens. Immune system exposure;

FIG. 5A shows how ASFV protein EP402R specifically causes porcine RBC aggregation, and FIG. 5B shows how ASFV protein E183L causes porcine RBC aggregation;

FIG. 6 is a detailed representation of the ASFV infection and replication cycle via RBC-mediated macrophage entry;

FIG. 7 is a detailed representation of strategy to block the cycle(s) shown in FIG. 6;

FIG. 8A and FIG. 8B shows how polyclonal antibodies raised against EP402R and E183L (p54) block these proteins from causing RBC aggregation;

FIG. 9A are graphs showing the effect of the deglycosylation of EP402R by PNGase F on porcine RBC aggregation and FIG. 9B shows the deglycosylation of EP402R and by PNGase F on a Coomassie stained gel;

FIG. 10A are sequence and structure diagrams showing the proposed regions and amino acid sequences of EP402R where dominant negative mutations will be made and screened to prevent RBC burrowing/binding resulting in exposure to the immune system. Each is derived from reference sequence China_AnhuiXCGQ_2018;

FIG. 10B is a representation of the orientation of folded full length EP402R in the outermembrane of ASFV;

FIG. 11A are sequence and structure diagrams showing the proposed regions and amino acid sequences of EP153R where dominant negative mutations will be made and screened to prevent RBC burrowing/binding resulting in exposure to the immune system. Each is derived from reference sequence China_AnhuiXCGQ_2018;

FIG. 11B is a representation of the orientation of folded full length EP153R in the outermembrane of ASFV;

FIG. 12A are sequence and structure diagrams showing the proposed regions and amino acid sequences of E183L where dominant negative mutations will be made and screened to prevent RBC burrowing/binding resulting in exposure to the immune system. Each is derived from reference sequence China_AnhuiXCGQ_2018;

FIG. 12B is a representation of the orientation of folded full length E183L in the capsid and viral envelope of ASFV.

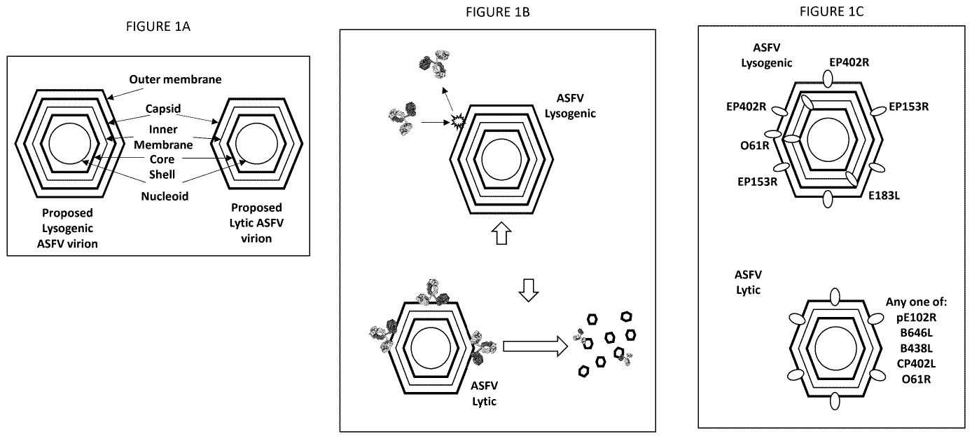

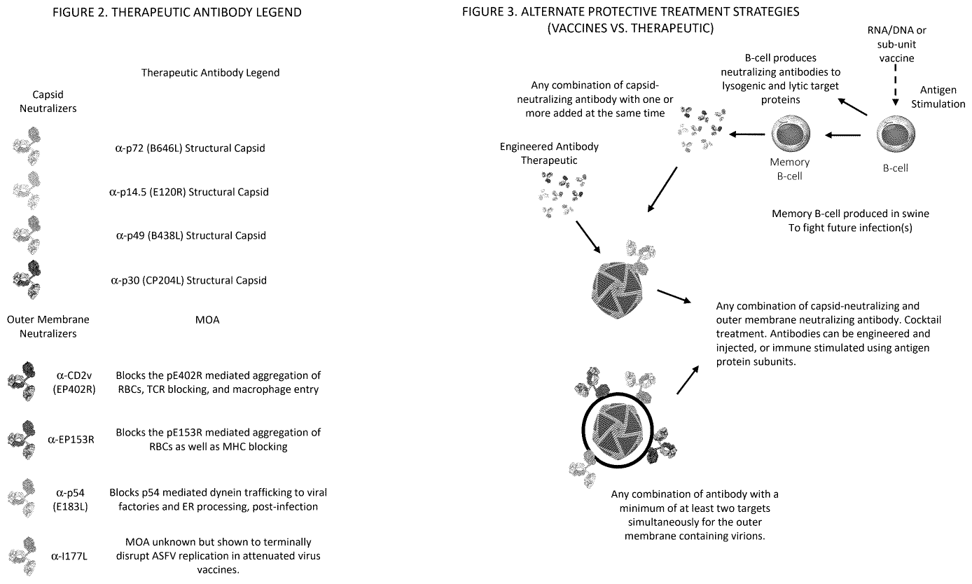

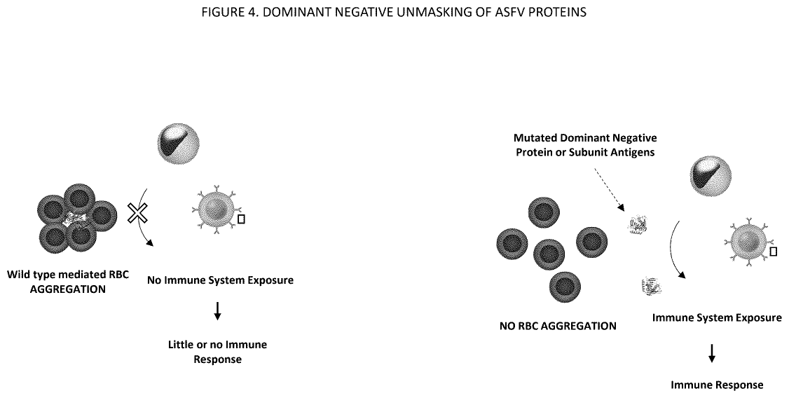

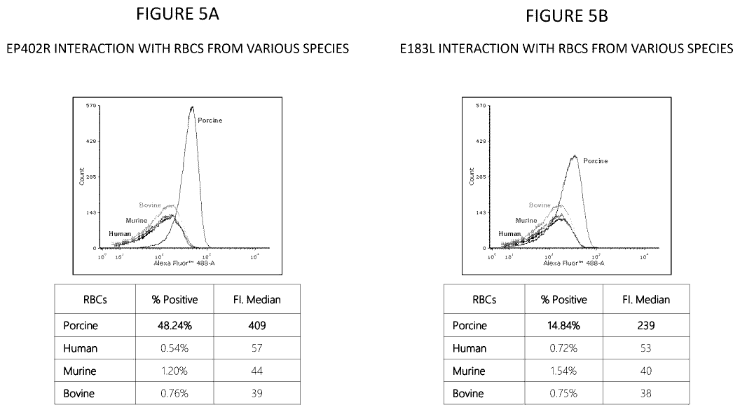

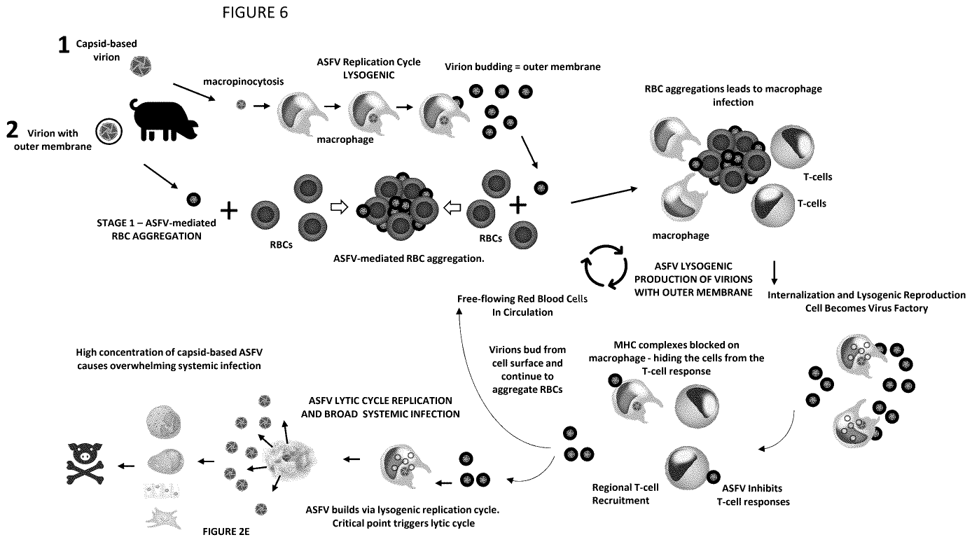

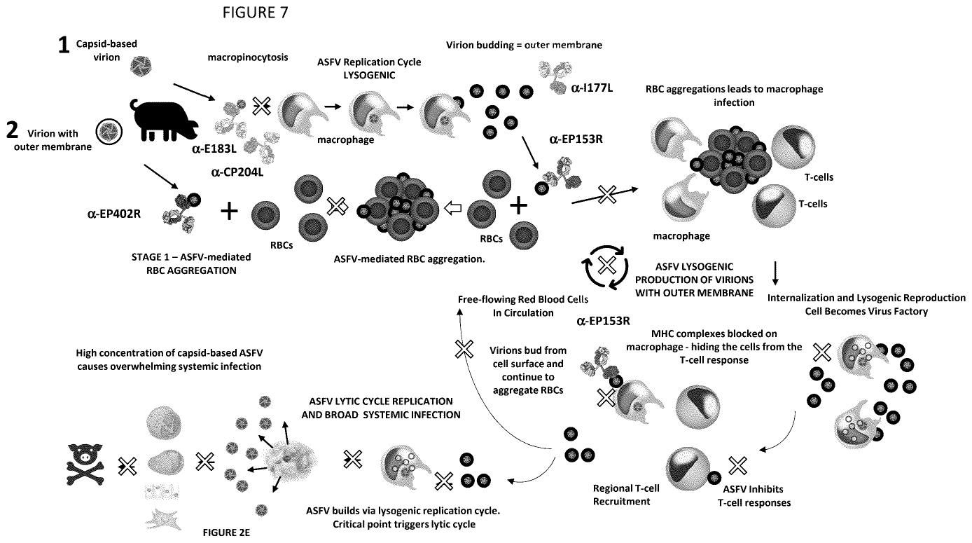

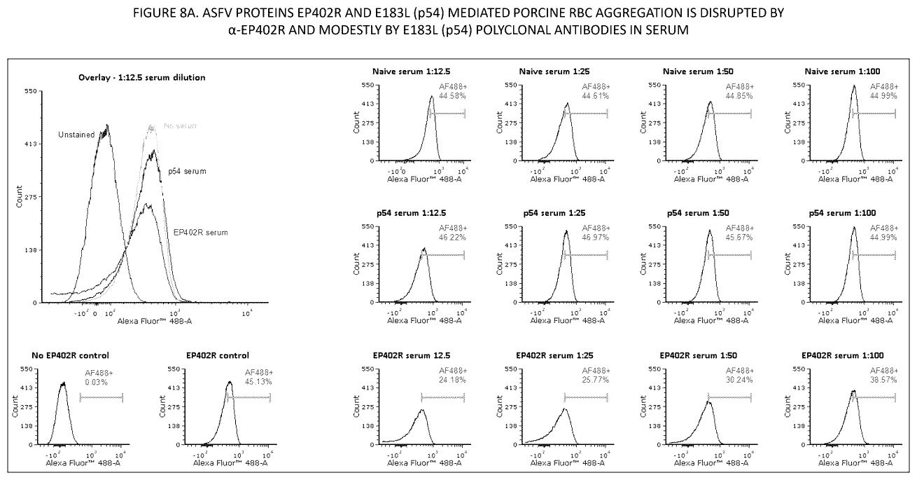

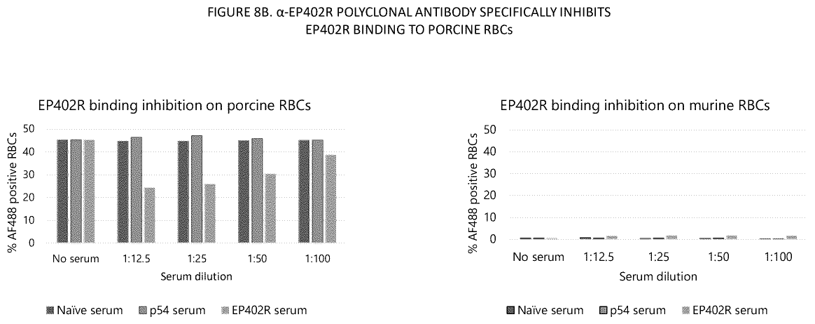

### DETAILED DESCRIPTION OF THE INVENTION

The present invention provides generally for compositions and methods of treating and/or preventing ASFV. The compositions include engineered ASFV outer-membrane protein antigen mutants (dominant negatives) that do not bind to RBCs yet elicit an antibody response that neutralizes wild type proteins present on infectious outer-membrane containing ASFV virions.

“Animal” as used herein refers to any non-human species of animal.

“Porcine” or “swine” as used herein, can be a domestic pig, wild boar, warthog, or bush pig.

The term “vector” includes cloning and expression vectors, as well as viral vectors and integrating vectors. An “expression vector” is a vector that includes a regulatory region. Vectors are also further described below.

The term “antibody” as used herein refers to a blood protein produced in response to and counteracting a specific antigen. Antibodies combine chemically with substances which the body recognizes as alien, such as bacteria, viruses, and foreign substances in the blood.

The term “m RNA” as used herein refers to a type of RNA in cells that carries genetic information required to make proteins.

The term “dominant negative” antigen for ASFV is used to describe a change to the native ASFV protein, so that the protein becomes exposed to the swine's immune system resulting in a strong immune response that can effectively inactivate the natural proteins on the virus.

Dominant negative antigens can be engineered several ways:

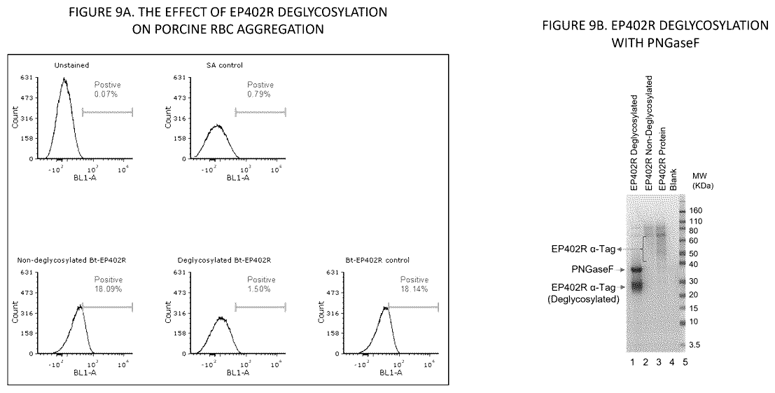

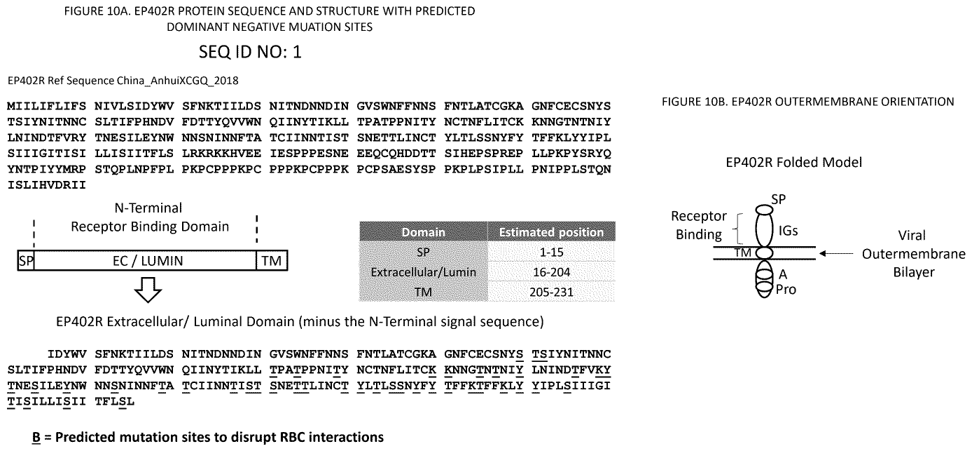

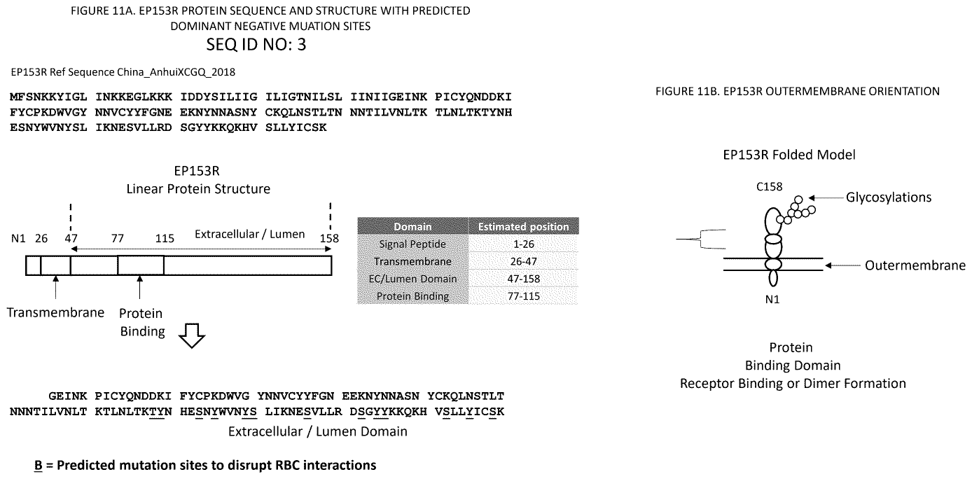

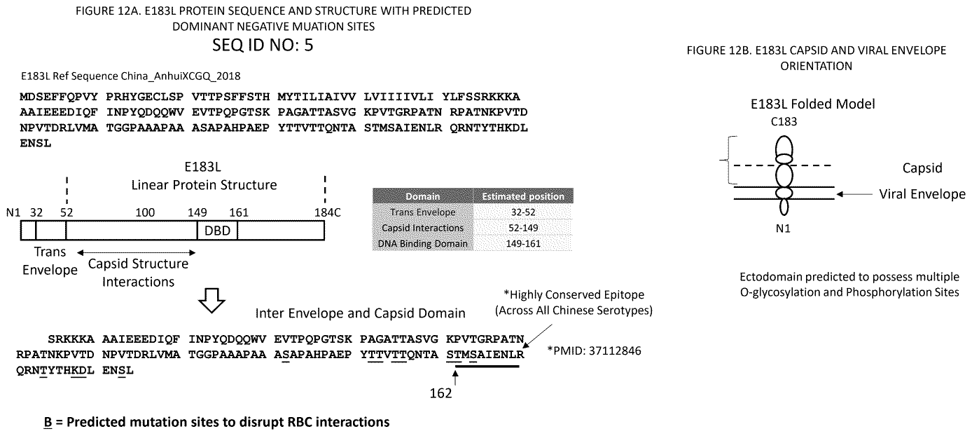

1) Mutations in the N- or O-glycosylation sites of each of the proteins pE402R (CD2v), EP153R, and EP183L (p54), respectively, to prevent receptor-mediated binding to RBCs. Applicants' data has shown that deglycosylation with PNGase F partially inhibits EP402R protein-mediated RBC aggregation (FIG. 9A). Therefore, silent mutations in these sites or sites that effect glycosylation that flank these sites, or multiple sites that prevent RBC-antigen receptor interactions can be implemented to create a dominant negative form of the antigen (FIGS. 10 through 12).

2) The dominant negative antigens can be screened using peptide libraries that define the epitope regions responsible for the RBC interaction. The epitopes responsible for binding can then be mutated to prevent binding. The mutations can be silent mutations to structure.

3) The dominant negative antigens can be screened using any type of applicable yeast two hybrid screening platform. The two-hybrid library can constitute a reticulocyte to protein antigen bait prey system.

4) High-throughput sequencing of libraries composed of fragments of yeast genes can identify polypeptide antigens with dominant negative properties (ref—PMID: 30962621).

5) By defining the receptors on the RBCs that interact with the outer-membrane antigens, the exact amino acids involved in the receptor-to-antigen interactions within the antigens can be resolved. RBC receptor resolution can be defined using multiple methods including two hybrid systems, crystallography, ligand linking systems, peptide interaction screening methods to name a few.

The dominant negative antigen can include a modification to the protein that prevents binding, such as; i) a structural component that sterically hinders the RBC interaction yet leaves critical amino residues exposed for immune recognition, ii) an alternate glycosylation (branched carbohydrate) that prevents binding, iii) a pegylated residue, iv) and other type of conjugated ligand that interferes with RBC binding without disrupting the structure of the protein so it maintains relevant immunoreactive epitopes that cross react with wild type virus.

The composition can include any degree of modification such as, but not limited to, 1%, 5%, 10%, 20%, 30%, 40%, 50%, 60%, 70%, 80%, 90%, or 100%.

A dominant negative antigen is necessary for a robust and efficacious antigen vaccine approach since the wild type antigens cause/lead to RBC aggregation and ‘hide’ from the immune response. Since capsid-based ASFV does not cause aggregation of RBCs, the use of dominant negative antigens is not necessary at this stage.

The present invention provides for a method of treating and/or preventing ASFV infection by administering a composition of a dominant negative antigen to an animal, preventing RBC aggregation from the antigen, and treating and/or preventing ASFV.

The present invention provides for a method of treating and/or preventing ASFV infection by administering a composition of a dominant negative antigen in combination with a wild type capsid protein or capsid subunit/domain/peptide to address both the lysogenic and lytic viral replication cycles, respectively.

The composition can be provided in a vaccine such as by 1) an mRNA vaccine for delivery to APCs and B-cells so that the mRNA expresses the antigen internally to create an immune response, 2) a direct antigen injection, or 3) a DNA vaccine (which sends instructions for making the antigen as DNA). Therefore, the present invention provides for a composition of a vaccine for ASFV, including a dominant negative antigen in a vaccine.

There can be additional applications for dominant negative proteins for viruses, bacteria, fungus, or parasites binding to reticulocytes or RBCs, which can be masked in other tissues. Hemagglutination is a tactic used by a handful of viruses to enhance their infectivity into target cells. For example, HIV-1 and HIV-2 bind to Duffy Antigen Receptor for Chemokines (DARC), a receptor on RBCs. The binding causes hemagglutination that in turn increases viral infectivity up to approximately 100-fold higher than unbound and circulating virus (Lachgar, et al., He and Neil, et al., Beck, et al.), the degree of which depends on the blood type of the infected individual (Abdulazeez, et al.). On the other hand, HIV-1 has also been shown to bind to P^{k} receptor of PBMCs to block competing viruses (Lund, et al.). Noroviruses also cause hemagglutination by selectively binding to group A, H, and/or difucosylated Lewis blood groups via Receptor/Le^{b} ligand interactions (Nillson, et al., Shirato-Horikoshi, et al.). Other examples of viruses that bind to RBCs and cause hemagglutination include Bovine Corona Virus (BCV), Porcine Hemagglutinating Encephalomyelitis Virus (PHEV), Influenza C, and Toroviruses (Zeng, et al.). Each of these RNA viruses cause hemagglutination by the binding of their Hemagglutinin Esterase (HE) protein to sialic acid containing receptors on RBCs. The HE protein has two functions: 1) binding to sialic acid containing receptors to cause agglutination. The aggregated RBCs then transport the virus to the target cells more efficiently than unbound and circulating virus. Once in contact with the target cell the HE protein 2) undergoes conformational change to activate its target cell receptor destroying properties, allowing the RBC to release its viral payload for cellular entry. However, these RNA viruses do not contain an outer-membrane or outer coat like ASFV. There is no outer-membrane to protect the inner core of the virus from an immune response. For example, PHEV is dominated by spike proteins that protrude from a single membrane that also contains the HE and other membrane proteins. Therefore, when the virus binds to RBCs via its HE proteins, its spike proteins are still exposed to antibody responses. There is no or little epitope hindrance. As such, antibodies against major PHEV proteins have been shown to be strongly neutralizing until the spike proteins (or others) mutate. Further, PHEV does not differentiate between species. It causes hemagglutination in mice, rats, chickens, and several other animals. Therefore, even though the agglutination of RBCs is a common tactic by some viruses to increase their infectivity to targeted cells, the modes of action are considerably different. ASFV is a DNA virus with much slower mutation rates caused by genetic drift or environmental mutagens, as opposed to many RNA viruses with higher mutations rates caused by lack of replicative proofreading. RNA viruses may not be masked as well by agglutinated RBCs due to their lack of a protective outer coat, but they make up for the difference by rapidly mutating to allow immune evasion. Taking into account these differences (especially the lack of species specificity in HE-containing RNA viruses), it is highly likely that ASFV binds specifically to swine RBCs by a different receptor, sialic acid motif, or carbohydrate than HE-containing RNA viruses.

The compositions described herein can be dosed in animals according to knowledge of those skilled in the art. The compositions can be in a concentration of 1-100% and include any suitable excipients such as further described below. Delivery routes can include, but are not limited to, epicutaneous, intradermal, subcutaneous, transdermal, intramuscular, intravenous, oral, transcorneal, intraocular, intracerebral, epidural, intrathecal, intraperitoneal, intraosseous, intranasal (such as into a snout of a pig), intratracheal, as well as other routes described further below.

The composition can be stored in various states that allow for shelf stability (one month to years) of proteins at various temperatures (4 degrees C, −20 degrees C, −20 to −80 degrees C), such as a lyophilized dry powder, aqueous solution, tris or phosphate buffers, or a solution including 25-50% glycerol or ethylene glycol that act as cryoprotectants.

The compound of the present invention is administered and dosed in accordance with good medical practice, taking into account the clinical condition of the individual animal, the site and method of administration, scheduling of administration, animal age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants, and vehicles. The compounds can be administered orally, subcutaneously, or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Chen et al. A seven-gene-deleted African swine fever virus is safe and effective as a live attenuated vaccine in pigs. Sci. China Life Sci. 2020, 63, 623-634.
2. Borca et al. Development of a highly effective African swine fever virus vaccine by deletion of the 1177L gene results in sterile immunity against the current epidemic Eurasia strain. J. Virol. 2020, 94, e02017-19.
3. Teklue et al, Generation and evaluation of an African swine fever virus mutant with deletion of the CD2v and UK genes. Vaccines 2020, 8, 763.
4. Liu et al. Cryo-EM structure of the African swine fever virus. Cell Host Microbe 2019, 26, 836-843.
5. Hübner et al. Efficient inhibition of African swine fever virus replication by CRISPR/Cas9 targeting of the viral p30 gene (CP204L). Sci Rep. 2018 Jan. 23; 8(1):1449.
6. Borca et al. CRISPR/Cas Gene Editing of a Large DNA Virus: African Swine Fever Virus. Bio Protoc. 2018 Aug. 20; 8(16):e2978.
7. Woźniakowski et al. Attempts at the Development of a Recombinant African Swine Fever Virus Strain with Abrogated EP402R, 9GL, and A238L Gene Structure using the CRISPR/Cas9 System. J Vet Res. 2020 Jun. 3; 64(2):197-205.
8. Petrovan et al. Role of African swine fever virus (ASFV) proteins EP153R and EP402R in reducing viral persistence in blood and virulence in pigs infected with BenindeltaDP148R. J Virol. 2021 October 13:JV10134021.
9. Yang et al. Identification of a new cell-penetrating peptide derived from the african swine fever virus CD2v protein. Drug Deliv. 2021 December; 28(1):957-962.
10. Chen et al. Porcine Immunoglobulin Fc Fused P30/P54 Protein of African Swine Fever Virus Displaying on Surface of S. cerevisiae Elicit Strong Antibody Production in Swine. Virol Sin. 2021 April; 36(2):207-219.
11. Sánchez et al. African swine fever virus uses macropinocytosis to enter host cells. PLoS Pathog. 2012; 8(6):e1002754.
12. Lachgar et al. Binding of HIV-1 to RBCs involves the Duffy antigen receptors for chemokines (DARC). Biomed Pharmacother. 1998; 52(10):436-9.
13. He et al, Duffy antigen receptor for chemokines mediates trans-infection of HIV-1 from red blood cells to target cells and affects HIV-AIDS susceptibility. Cell Host Microbe. 2008 Jul. 17; 4(1):52-62.
14. Beck et al. Human erythrocytes selectively bind and enrich infectious HIV-1 virions. PLoS One. 2009 Dec. 14; 4(12):e8297.
15. Abdulazeez et al. Carriage rate of Human Immunodeficiency Virus (HIV) infection among different ABO and Rhesus blood groups in Adamawa state, Nigeria. Biomedical Research (2008) Volume 19, Issue 1. Department of Biological Sciences, Federal University of Technology, Yola, Nigeria.
16. Lund et al. The human P(k) histo-blood group antigen provides protection against HIV-1 infection. Blood. 2009 May 14; 113(20):4980-91.
17. Nilsson et al. Norwalk virus-like particles bind specifically to A, H and difucosylated Lewis but not to B histo-blood group active glycosphingolipids. Glycoconj J. 2009 December; 26(9):1171-80.
18. Shirato-Horikoshi et al. Binding activity of norovirus and sapovirus to histo-blood group antigens. Arch Virol. 2007; 152(3):457-61.
19. Zeng et al. Structure of coronavirus hemagglutinin-esterase offers insight into corona and influenza virus evolution. Proc Natl Acad Sci USA. 2008 July 1; 105(26):9065-9.

What is claimed is:

1. A composition comprising engineered ASFV outer-membrane protein antigen mutants (dominant negatives) that do not bind to RBCs and elicit an antibody response that neutralizes wild type proteins present on infectious outer-membrane containing ASFV virions.

2. A composition as set forth in claim one including the dominant negatives further defined as an antigen for ASFV used to describe a change to the native ASFV protein, so that the protein becomes exposed to the swine's immune system resulting in a strong immune response that can effectively inactivate the natural proteins on the virus.

3. A method of treating and/or preventing ASFV, including the steps of:
administering a composition of a dominant negative antigen to an animal;
preventing RBC aggregation from the antigen; and
treating and/or preventing ASFV.

4. A method as set forth in claim two wherein defined as an antigen for ASFV used to describe a change to the native ASFV protein, so that the protein becomes exposed to the swine's immune system resulting in a strong immune response that can effectively inactivate the natural proteins on the virus.

5. A method of engineering a dominant negative antigen by the steps engineering mutations in the N- or O-glycosylation sites of each of the proteins pE402R (CD2v), EP153R, and EP183L (p54), respectively, and preventing receptor-mediated binding to RBCs whereby the silent mutations in these sites can be implemented to create a dominant negative form of the antigen.

6. A method of engineering dominant negative antigens by screening the dominant negative antigens using peptide libraries that define the epitope regions responsible for the RBC interaction whereby the epitopes responsible for binding can then be mutated to prevent binding.

7. The method of claim 6 wherein the mutations can be silent mutations to structure.

8. A method of engineering dominant negative antigens by screening dominant negative antigens using any type of applicable yeast two-hybrid screening platform, the two-hybrid screening platform constituting a reticulocyte to protein antigen bait-prey system.

9. A dominant negative antigen including a modification to the protein that prevents binding, such as; i) a structural component that sterically hinders the RBC interaction yet leaves critical amino residues exposed for immune recognition, ii) an alternate glycosylation (branched carbohydrate) that prevents binding, iii) a pegylated residue, iv) and other type of conjugated ligand that interferes with RBC binding without disrupting the structure of the protein so it maintains relevant immunoreactive epitopes that cross react with wild type virus.

10. A composition of a vaccine for ASFV, comprising a dominant negative antigen in a vaccine.
